(19) United States
(12) Patent Application Publication
(10) Pub. No.: US 2012/0320339 A1
(43) Pub. Date: Dec. 20, 2012

(54) OPHTHALMOLOGIC APPARATUS, OPHTHALMOLOGIC SYSTEM, CONTROLLING METHOD FOR OPHTHALMOLOGIC APPARATUS, AND PROGRAM FOR THE CONTROLLING METHOD

(75) Inventor: Keiko Yonezawa, Kawasaki-shi (JP)

(73) Assignee: CANON KABUSHIKI KAISHA, Tokyo (JP)

(21) Appl. No.: 13/488,735

(22) Filed: Jun. 5, 2012

(30) Foreign Application Priority Data
Jun. 14, 2011 (JP) 2011-132328

(51) Int. Cl. A61B 3/15

(52) U.S. Cl. 351/208; 351/246

(57) ABSTRACT

Provided is an image processing apparatus capable of solving a problem that a curvature of an acquired retina image varies in accordance with a working distance (WD) when the retina image is acquired by an OCT apparatus. The image processing apparatus includes: an image acquiring unit configured to acquire a tomographic image of a fundus of an eye to be inspected; a calculating unit configured to calculate a working distance, based on a predetermined layer of the tomographic image, a coherence gate position, and an axial length of the eye to be inspected, at a time when the tomographic image is acquired; and a correcting unit configured to correct the tomographic image based on the working distance.

### BACKGROUND OF THE INVENTION

1. Field of the Invention

The present invention relates to an ophthalmologic apparatus, an ophthalmologic system, a controlling method for an ophthalmologic apparatus, and a program for the controlling method. In particular, the present invention relates to an image processing apparatus and an image processing method which are suitable for those ophthalmologic apparatuses used in ophthalmological diagnosis and treatment.

2. Description of the Related Art

For the purpose of early diagnosis of diseases ranking high in causes of lifestyle related diseases or loss of eyesight, fundus inspection has been performed widely. An optical tomographic imaging apparatus (or optical coherence tomography (OCT) apparatus) that acquires a fundus tomographic image by utilizing optical interference enables three-dimensional observation of the state of an internal structure of a retinal fundus, and hence the OCT apparatus is useful for performing diagnosis of the diseases. The retinal fundus has a multiple layer structure, and it is known that a thickness of each layer can be used as an index indicating development of the disease. The OCT apparatus has enabled quantitative observation of the layer structure of the retinal fundus, and hence it is expected that the development of the disease can be grasped more precisely.

In recent years, OCT observation of myopic eyes that are common in Asia has received attention. It is known that curvature of retina is larger in a myopic eye than in a fundus of an eye that is not a myopic eye in some cases, and correlation between the curvature of retina and a disease has been gaining attention.

FIG. 3 is a schematic view of a tomographic image of a macula lutea and its vicinity of a myopic eye fundus. FIG. 3 illustrates boundaries L1 to L4 of a layer structure of a retina. The boundary L1 is a boundary between an internal limiting membrane and its upper organism (hereinafter, referred to as ILM), the boundary L2 is a boundary between a nerve fiber layer and its lower layer (hereinafter, referred to as NFL), the boundary L3 is a boundary between a photoreceptor inner/outer segment junction and its upper layer (hereinafter, referred to as IS/OS), and the boundary L4 is a boundary between a retinal pigment epithelium and its lower organism (hereinafter, referred to as RPE). As illustrated in FIG. 3, it is known that a thickness of the retina layer is generally smaller than the actual thickness thereof in a tomographic image of a fundus having a large curvature due to myopia or the like.

When an image of a retina is acquired by the OCT apparatus, a distance from an objective lens of the OCT apparatus to an eye whose image is to be acquired is called a working distance (WD). This distance is designated to a value optimal for the apparatus, and an operation method is determined so that a focal point obtained by using an anterior eye part becomes substantially close to this optimal value. However, in a case of an eye having a large axial length and a large curvature as in a myopic eye, the image may be often acquired at a point shifted from the optimal value in order that the retina layers are placed within one tomographic image.

When an image is acquired by the OCT apparatus, if the WD changes, the curvature of retina changes in the acquired tomographic image. Quantitative observation of the curvature is necessary in observation of a myopic eye, and hence it is necessary to correct the curvature.

US 2007/0076217 discloses measurement of an axial length of an eye to be inspected based on a tomographic image of an anterior eye part and a tomographic image of a fundus of the eye to be inspected, which are obtained by two OCT apparatuses.

However, although the axial length can be measured accurately through the method disclosed in U.S. Pat. No. 2007/0076217, the apparatus including two mirror systems becomes larger in size because two OCT apparatuses are necessary.

Incidentally, in order to correct the above-mentioned curvature, it is necessary to measure the WD every time one OCT tomographic image is acquired. Therefore, as described in US 2007/0076217, the axial length is measured mechanically every time one OCT tomographic image is acquired. This means that speed of acquiring one OCT tomographic image is restricted by the driving speed of a reference mirror. Otherwise, it is necessary to use a reference mirror that can be driven at such a high speed that one OCT tomographic image can be acquired.

### SUMMARY OF THE INVENTION

It is a purpose of the present invention to provide an ophthalmologic apparatus, an ophthalmologic system, a controlling method for an ophthalmologic apparatus, and a program for the controlling method, which are suitable for solving the above-mentioned problem.

In order to solve the above-mentioned problem, the present invention provides an ophthalmologic apparatus, including: an image acquiring unit configured to acquire a tomographic image of a fundus of an eye to be inspected; a calculating unit configured to calculate a working distance, based on a predetermined layer of the tomographic image, a coherence gate position, and an axial length of the eye to be inspected, at a time when the tomographic image is acquired; and a correcting unit configured to correct the tomographic image based on the working distance.

According to the present invention, a curvature of a retina can be corrected based on information obtained by analyzing a tomographic image. Thus, it becomes possible to perform quantitative analysis of the curvature in a myopic eye, and it also becomes possible to perform observation with time and comparison among eyes to be inspected.

Further features of the present invention will become apparent from the following description of exemplary embodiments with reference to the attached drawings.

### BRIEF DESCRIPTION OF THE DRAWINGS

FIG. 1 is a diagram illustrating a functional configuration of an image processing apparatus according to a first embodiment of the present invention.

FIGS. 2A and 2B are flowcharts illustrating a process procedure of the image processing apparatus according to the first embodiment of the present invention.

FIG. 3 is a schematic view of a retina tomographic image having a large curvature.

FIG. 4 is a diagram illustrating an OCT apparatus.

FIG. 5A is a diagram illustrating a positional relationship among a WD, an eyeball, and a retina in an acquired image.

FIG. 5B is a schematic diagram illustrating an outline of ray tracing for determining a rotation center.

FIG. 5C is a schematic diagram illustrating coordinate conversion.

FIG. 6A shows a variation of curvature on an image due to a variation of working distance in a model eye.

FIG. 6B shows an example of curvature correction by the process according to the first embodiment of the present invention.

FIG. 7A shows an image obtained by superimposing the images shown in FIG. 6A.

FIG. 7B shows an image obtained by superimposing the images shown in FIG. 6B.

FIG. 8 is a diagram illustrating a functional configuration of the image processing apparatus according to a second embodiment of the present invention.

FIG. 9 is a flowchart illustrating a process procedure of the image processing apparatus according to the second embodiment of the present invention.

FIG. 10 is a table showing a model of refraction elements of an eye to be inspected.

FIG. 11 is a graph showing an example of a relationship between the working distance and a distance from the rotation center to the retina.

FIG. 12 is a diagram illustrating a functional configuration of the image processing apparatus according to a third embodiment of the present invention.

FIG. 13 is a flowchart illustrating a process procedure of the image processing apparatus according to the third embodiment of the present invention.

### DESCRIPTION OF THE EMBODIMENTS

Preferred embodiments of the present invention will now be described in detail in accordance with the accompanying drawings.

### First Embodiment

In a first embodiment of the present invention, when performing quantitative measurement of curvature of retina of a diseased eye such as a myopic eye that is known to have a large curvature, variation of curvature of retina of the acquired tomographic image is corrected by using a working distance (WD) obtained when the image is acquired so that a correct curvature is measured. More specifically, an axial length of an eye to be inspected is obtained, the WD is calculated from a coherence gate position when the image is acquired and a retina position in an acquired tomographic image, and the acquired tomographic image is corrected based on this WD value. With this correction, a correct curvature can be measured, and comparison among eyes to be inspected or evaluation of the variation with time can be performed.

In other words, a distance from a reference point to an objective lens is obtained, and the axial length of the eye to be inspected, the coherence gate position when the image is acquired, and the retina position of the acquired tomographic image are obtained. Thus, the WD is calculated, and hence the curvature is corrected.

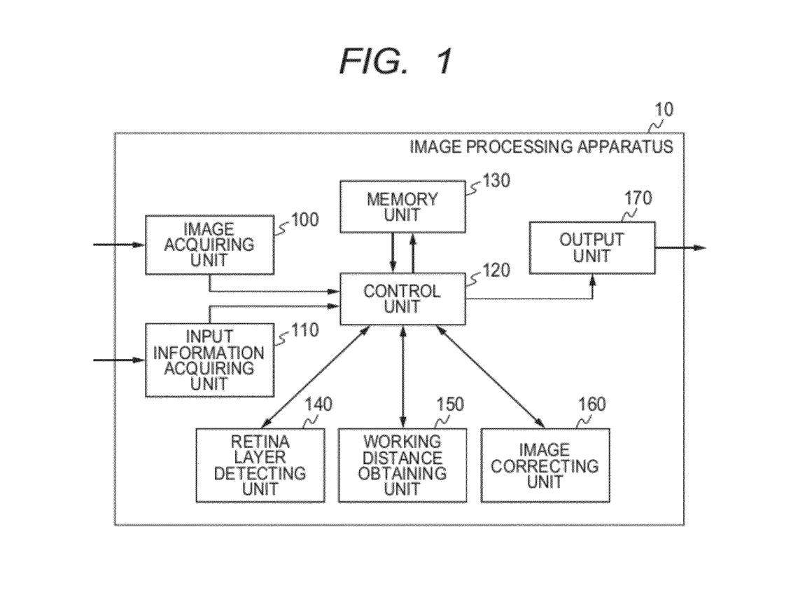

FIG. 1 is a diagram illustrating a functional configuration of an image processing apparatus 10 according to this embodiment. In FIG. 1, an image acquiring unit 100 corresponds to an image acquiring unit of the present invention, which acquires a tomographic image acquired by an optical tomographic imaging apparatus (OCT apparatus) or a tomographic image stored in an external database directly or via a network or the like. An input information acquiring unit 110 acquires, from the OCT apparatus or the database, information of the axial length of the eye to be inspected and the coherence gate position when the image is acquired. The acquired information is stored in a memory unit 130 via a control unit 120. The image acquiring unit 100 further includes a retina layer detecting unit 140, a working distance obtaining unit 150, and an image correcting unit 160. Based on the obtained working distance, the entire image is corrected so that curvature of a detected layer in the acquired tomographic image is corrected, and a result of the correction is stored in the memory unit 130. An output unit 170 outputs the corrected tomographic image to a monitor or the like, and saves the process result stored in the memory unit 130 in the database.

FIG. 4 is a diagram illustrating a configuration of the optical tomographic imaging apparatus used in this embodiment. The optical tomographic imaging apparatus is constituted of a Michelson interferometer. Emerging light 102 from a light source 101 is guided to a single mode fiber 107 to enter an optical coupler 108. The optical coupler 108 split the light into reference light 103 and measuring light 104. Then, the measuring light 104 is reflected or scattered by a measurement point of a retina 125 to be observed and becomes return light 105 to travel back to the optical coupler 108. Then, the optical coupler 108 combines the return light 105 with the reference light 103 that has propagated through a reference light path, so as to emit combined light 106, which then reaches a spectroscope 119.

The light source 101 is a super luminescent diode (SLD), which is a typical low coherence light source. As to the wavelength, near infrared light is suitable in view of measuring an eye. Further, the wavelength affects a lateral resolution of the acquired tomographic image, and hence the wavelength is desirable to be as short as possible. In this embodiment, a center wavelength is set to 840 nm, and a wavelength width is set to 50 nm. It is to be understood that another wavelength may be selected depending on a measurement part to be observed. Note that, as a type of the light source, an SLD is selected in this embodiment, but any type of light source may be selected as long as the light source can emit low coherent light, and an amplified spontaneous emission (ASE) light source or the like may also be used.

Next, the reference light path of the reference light 103 is described. The reference light 103 split by the optical coupler 108 is converted into substantially parallel light by a lens 109-1, and is then emitted. Next, the reference light 103 passes through a dispersion compensation glass 310, and the direction of the reference light 103 is changed by a mirror 111. Then, the reference light 103 is guided to the spectroscope 119 again via the optical coupler 108. Note that, the dispersion compensating glass 310 compensates for dispersion of the measuring light 104 propagating forward and backward between an eye to be inspected 124 and a scanning optical system with respect to the reference light 103. In this embodiment, as a diameter of an eyeball of an average Japanese, a typical value of 24 mm is supposed. An optical path length of the reference light can be adjusted by moving the mirror 111 in the arrow direction by an electric stage 112 so that the coherence gate position corresponding to this optical path length can be adjusted. The coherence gate means a position in the measuring light path which matches the reference light path in terms of the distance. The electric stage 112 is controlled by the control unit 120, and the control unit 120 stores position information of the electric stage 112 when the image is acquired and the acquired tomographic image associated with each other.

Next, the measuring light path of the measuring light 104 is described. The measuring light 104 split by the optical coupler 108 is converted into substantially parallel light by the lens 109-2, and is then emitted. The resultant light is input to a mirror of an XY scanner 113 constituting the scanning optical system. FIG. 4 illustrates the XY scanner 113 as one mirror, but in reality, two mirrors are disposed closely to each other, which include an X-scan mirror and a Y-scan mirror. The measuring light passes through a lens 114 and an objective lens 128 to reach the eye to be inspected 124. The measuring light 104 that has reached the eye to be inspected 124 is reflected by the retina 125 and the like to be the reflected light 105, which propagates backward through the path of the measuring light 104 and enters the optical coupler 108, so as to be combined with the reference light 103.

In addition, the combined light 106 generated by the optical coupler 108 is split into beams having individual wavelengths by the spectroscope 119, and intensities of the respective beams are detected and output to a computer 320. Then, the computer 320 performs a process of Fourier transform or the like so as to generate a tomographic image. Note that, the memory unit of the computer 320 stores design values of the OCT apparatus, which can be output externally.

Next, the working distance (WD) is described. Herein, a WD 126 is defined as a distance from a surface of a cornea 122 to a surface of the objective lens 128. First, in a general OCT optical system, a value of the WD 126 is designed so that a pupil 129 of the eye becomes a rotation center when the measuring light 104 scans the retina 125. Therefore, it is desirable to adjust the WD 126 to be a design value so as to acquire the tomographic image. However, the OCT optical system has a small NA and therefore a large focal depth. As a result, even if the WD is shifted from the design value, the image can be acquired. Note that, if the WD is shifted largely from the design value, light may be blocked by an iris 127, or the image may be defocused.

In this embodiment, the image processing apparatus 10 of FIG. 1 may acquire the tomographic image directly from the computer 320 of the optical tomographic imaging apparatus of FIG. 4, or may acquire the tomographic image via a network. In the latter case, the tomographic image acquired by the optical tomographic imaging apparatus and the information of the eye to be inspected are stored in the database connected via network, and the image processing apparatus 10 acquires the tomographic image and the information of the eye to be inspected from the database.

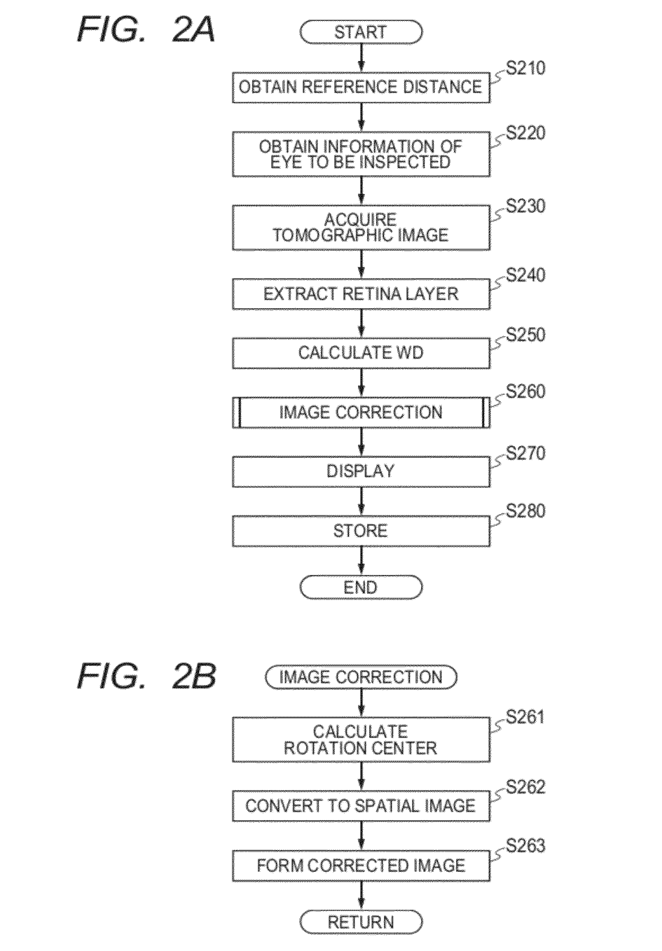

Next, referring to a flowchart of FIG. 2, a process procedure of the image processing apparatus 10 of this embodiment is described.

(Step S210)

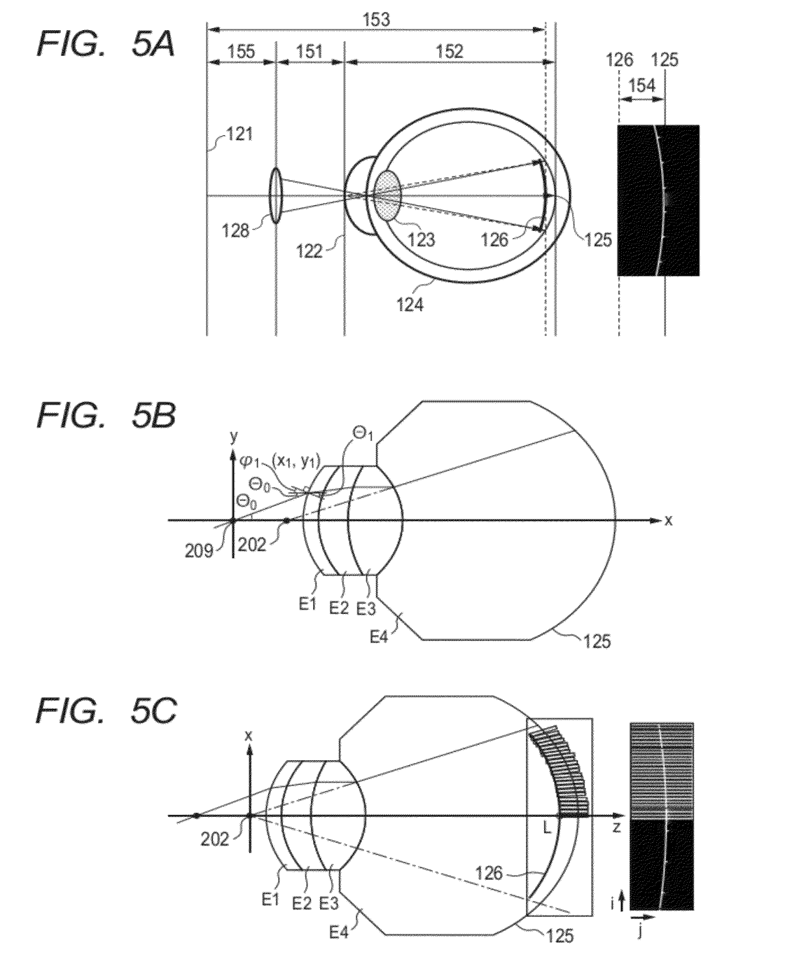

In Step S210, the input information acquiring unit 110 acquires information about the OCT apparatus, for example, a reference distance 155 that is a distance from a reference position 121 to the objective lens 128, which is illustrated in FIG. 5A, from the computer 320 and stores the information in the memory unit 130 via the control unit 120. Referring to FIG. 5A, the reference position 121 of acquiring the image is now described in more detail. The reference position 121 of acquiring the image in this embodiment is a coherence gate position when the mirror 111 moves to the origin (a position within a moving range of the mirror 111 at which the reference light path length becomes shortest). The distance 155 from the reference position 121 to the objective lens is fixed and set to be always a determined value. In FIG. 5A, the reference position 121 is illustrated on the opposite side to the eyeball when viewed from the objective lens 128 for easy understanding, but in reality, the reference position 121 exists on the same side as the eyeball when viewed from the objective lens 128. This is because that it is sufficient if the moving range of the mirror 111 is set so that the coherence gate position covers an assumed range of the object to be measured. Therefore, if the positional relationship is defined as illustrated in FIG. 5A, the reference distance 155 becomes a negative value.

Note that, the reference position 121 is not limited to the position obtained in the above-mentioned case, but may be a position obtained in a case where the mirror 111 is placed in an arbitrary position. The reference position 121 is adjusted so that the origin is a mirror position when the reference light path length becomes a certain value, and hence the constant reference distance 155 can be set without depending on the apparatus.

(Step S220)

In Step S220, the input information acquiring unit 110 as an eye information acquiring unit in the present invention acquires information of the eye to be inspected from the database or an input by an operator using an input unit (not shown). Herein, information of the eye to be inspected refers to eye parameters typified by an axial length, which are characteristics unique to the eye to be inspected. The acquired information is stored in the memory unit 130 via the control unit 120. In other words, the eye information acquiring unit acquires eye parameters such as a distance from the reference position 121 to the coherence gate position, the axial length of the eye to be inspected, and the like. Note that, the light beam is refracted and propagates inside the eye to be inspected as a property of light. Therefore, through use of a refractive index and a curvature of the anterior eye part (such as the cornea and a crystalline lens) in addition to the axial length, the above-mentioned property of light can be taken into account, and hence an accuracy of correcting the tomographic image can be improved.

(Step S230)

In Step S230, the image acquiring unit 100 acquires the tomographic image to be analyzed from the optical tomographic imaging apparatus of FIG. 4 connected to the image processing apparatus 10, or the database storing the tomographic image acquired by the optical tomographic imaging apparatus. The acquired tomographic image is stored in the memory unit 130 via the control unit 120.

In addition, in this step, the coherence gate position 126 when the acquired tomographic image is acquired and is stored in the memory unit 130 via the control unit 120. The coherence gate position 126 may be described in an image acquiring information file attached to the tomographic image, or may be included as tag information of the image. In addition, the reference position 121 is the coherence gate position when the mirror 111 is at the origin, and hence when the moving distance from the origin of the coherence gate is converted into an actual distance length based on a value of the coherence gate position 126 when the image is acquired, and a distance 153 is obtained as illustrated in FIG. 5A.

(Step S240)

In Step S240, the retina layer detecting unit 140 detects a retina layer boundary from the tomographic image stored in the memory unit 130. The retina layer detecting unit 140 functions as a layer extracting unit in the present invention, which extracts a predetermined layer from the tomographic image. There are known various methods as a layer segmentation method. In this embodiment, description is given of a case where an edge to be a layer boundary is extracted by using an edge enhancing filter, and then the detected edge and a layer boundary are associated with each other by using medical knowledge about the retina layer. The retina position for measuring the axial length is generally the ILM, and hence detection of the RPE having higher luminance than the ILM is described in this embodiment, but other layer boundaries can also be detected by the same method.

First, the retina layer detecting unit 140 performs a smoothing filter process on the tomographic image so as to remove noise components. Then, an edge detection filter process is performed so that an edge component is detected from the tomographic image, and then an edge corresponding to a boundary between layers is extracted. Further, a background region is specified from the tomographic image on which the edge detection has been performed, and a luminance value characteristic of the background region is extracted from the tomographic image. Then, a peak value of the edge component and the luminance value characteristic between the peaks are used so that the boundary between the layers is determined.

For instance, the retina layer detecting unit 140 searches for an edge from a corpus vitreum side in a depth direction of the fundus, and a boundary between the corpus vitreum and the retina layer (ILM) is determined from a peak of the edge component, luminance characteristics of the upper and lower parts thereof, and a luminance characteristic of the background region. Further, an edge is searched for in the depth direction of the fundus, and a retina pigment epithelium (RPE) layer boundary is determined by referring to the peak of the edge component, the luminance characteristic between the peaks, and a luminance characteristic of the background region. Through the process described above, a boundary between layers can be detected.

The ILM boundary (control point) detected in this way is sent to the control unit 120 and is stored in the memory unit 130.

Here, a height (y coordinate) of an ILM boundary position 302 in a center 301 of the acquired image is converted into an actual distance by using a pixel resolution and a refractive index and is stored in the memory unit 130 via the control unit 120.

(Step S250)

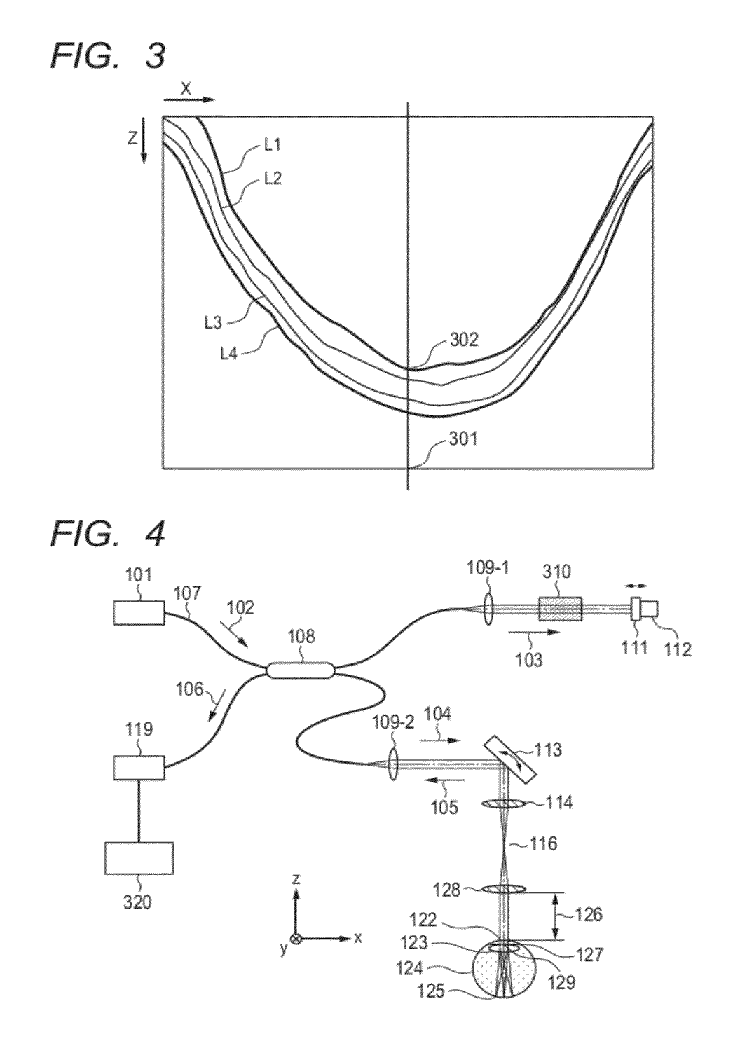

In Step S250, the working distance obtaining unit 150 acquires the reference distance 155, an axial length 152, and the coherence gate distance 153 acquired in Steps S210 to S230 from the memory unit 130. The working distance obtaining unit 150 functions as a calculating unit in the present invention that calculates the working distance as a distance to the eye to be inspected when the tomographic image is acquired based on a distance to the coherence gate position, an axial length of the eye to be inspected, and a retina distance. In addition, the control point of the retina position detected in Step S240 is acquired from the memory unit 130, and an image retina distance 154 to the retina position in the image is calculated. The calculation of the image retina distance 154 from the coherence gate when the image is acquired to the extracted layer is performed in the unit defined as the obtaining unit in the above-mentioned configuration. Specifically, as illustrated in FIG. 3, the ILM position 302 at the image center 301 is acquired, and a z coordinate thereof is determined so that the number of pixels from the upper limit of the image to the retina position is obtained. Based on the pixel resolution of the image and the refractive index of the corpus vitreum, the actual distance length is obtained by conversion, and hence the image retina distance 154 of FIG. 5A is acquired.

FIG. 5A illustrates a positional relationship among those distances. That is, the following equation is satisfied.

(coherence gate position distance 153)+(image retina distance 154)=(reference distance 155)+(working distance 151)+(axial length 152) (Equation 0)

It is now supposed that the image is generated so that the upper limit part of the image becomes the coherence gate 126. If there is a difference ΔL between the upper limit part of the image and the coherence gate position, a value obtained by subtracting the difference ΔL from the reference distance 155 is set as a new reference distance, so as to perform the same calculation. From Equation 1, the working distance 151 can be acquired.

The working distance 151 acquired in this way is stored in the memory unit 130 via the control unit 120.

(Step S260)

In Step S260, the image correcting unit 160 corrects the acquired image based on the working distance acquired in Step S250. The process in Step S260 is divided into three parts as illustrated in FIG. 2B, which are calculation of the rotation center in Step S261, conversion into a spatial image in Step S262, and generation of a corrected image in Step S263. Individual steps are described in detail as follows.

(Step S261)

In Step S261, the image correcting unit 160 performs calculation of the rotation center based on the working distance acquired in Step S250. The image correcting unit 160 corresponds to a correcting unit in the present invention, which corrects the tomographic image based on the determined working distance.

FIG. 5B is a schematic diagram illustrating an outline of ray tracing for determining the rotation center.

FIG. 5B illustrates a cornea E1, an anterior chamber E2, a crystalline lens E3, and a corpus vitreum E4. FIG. 5B further illustrates a rotation center 209 viewed from the objective lens and an effective rotation center 202 viewed from the retina. All rays entering at an angle of θ₀ from the rotation center 209 are refracted by a cornea surface, a boundary between the cornea and the anterior chamber, a boundary between the anterior chamber and the crystalline lens, and a boundary between the crystalline lens and the corpus vitreum, which are approximated by spheres, and enter the retina. The rays entering from the rotation center 209 look as if the rays enter from the effective rotation center 202 when viewed from the retina.

Parameters including a curvature radius, a thickness, and a refractive index are assigned to each of the cornea, the anterior chamber, the crystalline lens, and the corpus vitreum. Average parameter values of human eyes are shown in FIG. 10. In addition, as parameters of the model eye, values designed for each model eye are used.

The ray tracing is performed as follows. First, rays entering from a pivot position at an angle of θ₀ are expressed by the following equation, supposing that the pivot position is the origin.

y=x tan θ₀ Equation 1

The cornea surface is expressed by the following equation, supposing that an x value of a point where the cornea surface crosses an x axis is denoted by L₁, and a curvature radius of the cornea surface is denoted by R₁.

(x−L₁−R₁)²+y²=R₁² Equation 2

An intersection point (x₁, y₁) where the incident light crosses the cornea surface is determined by Equations 1 and 2.

Supposing that an angle between the direction perpendicular to the cornea surface and the incident ray at the intersection point is θ₀+φ₁, the angle φ₁ is determined as follows.

y₁/R₁=sin θ₁ Equation 3

In addition, refraction at the cornea surface is expressed as follows by Snell's law, supposing that an angle between a ray after the refraction and the direction perpendicular to the cornea surface is denoted by θ₁.

n₀ sin(θ₀+φ₁)=n₁ sin(θ₁) Equation 4

In Equation 4, n denotes a refractive index of each medium, and it is supposed that a refractive index n₀ in the air is 1 (one).

Next, a case where the ray refracted at the cornea enters the boundary between the cornea and the anterior chamber is described. The ray refracted at the cornea surface is expressed as a line that passes the intersection point (x₁, y₁) and has a gradient of tan(θ₁−φ₁). The boundary between the cornea and the anterior chamber is expressed by the following equations, supposing that an x value of the point where the boundary between the cornea and the anterior chamber crosses the x axis is denoted by L₂, and the curvature radius of the boundary is denoted by R₂, similarly to the case of Equation 2.

y−y₁=(x−x₁)tan(θ₁−φ₁) Equation 5

(x−L₂−R₂)²+y²=R₂² Equation 6

After that, in the same manner, an intersection point (x₂, y₂) of the incident light with the boundary between the cornea and the anterior chamber is determined from Equations 5 and 6. Supposing that an angle between the incident ray and a direction perpendicular to the boundary between the cornea and the anterior chamber at the intersection point is θ₁−φ₁+φ₂, the angle φ₂ is determined as follows.

y₂/R₂=sin φ₂ Equation 7

From the Snell's law, refraction at the boundary between the cornea and the anterior chamber is expressed as follows, supposing that an angle between the ray after the refraction and the direction perpendicular to the boundary between the cornea and the anterior chamber is denoted by θ₂.

n₁ sin(θ₁−φ₁+φ₂)=n₂ sin(θ₂) Equation 8

In this way, refractions at the cornea surface, at the boundary between the cornea and the anterior chamber, at the boundary between the anterior chamber and the crystalline lens, and at the boundary between the crystalline lens and the corpus vitreum, which are all approximated by spheres, are determined, and hence ray tracing to the retina can be performed. In this embodiment, when the intersection point between the ray and the interface is determined, each solution of Equations 1, 2, 5, and 6 is an intersection point of a circle and a line, which include two points. An appropriate one of the points is selected considering a shape of the eyeball.

In addition, the ray entering the retina is expressed as follows as a line that passes an intersection point (x₄, y₄) at the boundary between the crystalline lens and the corpus vitreum and has a gradient of tan(θ₄−Φ₄).

y−y₄=(x−x₄)tan(θ₄−φ₄) Equation 9

When viewed from the retina, it looks as if the scan is performed with, as the rotation center, the intersection point (xₚ, 0) of the line and the x axis. Therefore, this intersection point is set as the effective rotation center 202.

$${x}_{p} = {x}_{4} + \frac{{y}_{4}}{tan ( {θ}_{4} - {φ}_{4} )} \qquad Equation 10$$

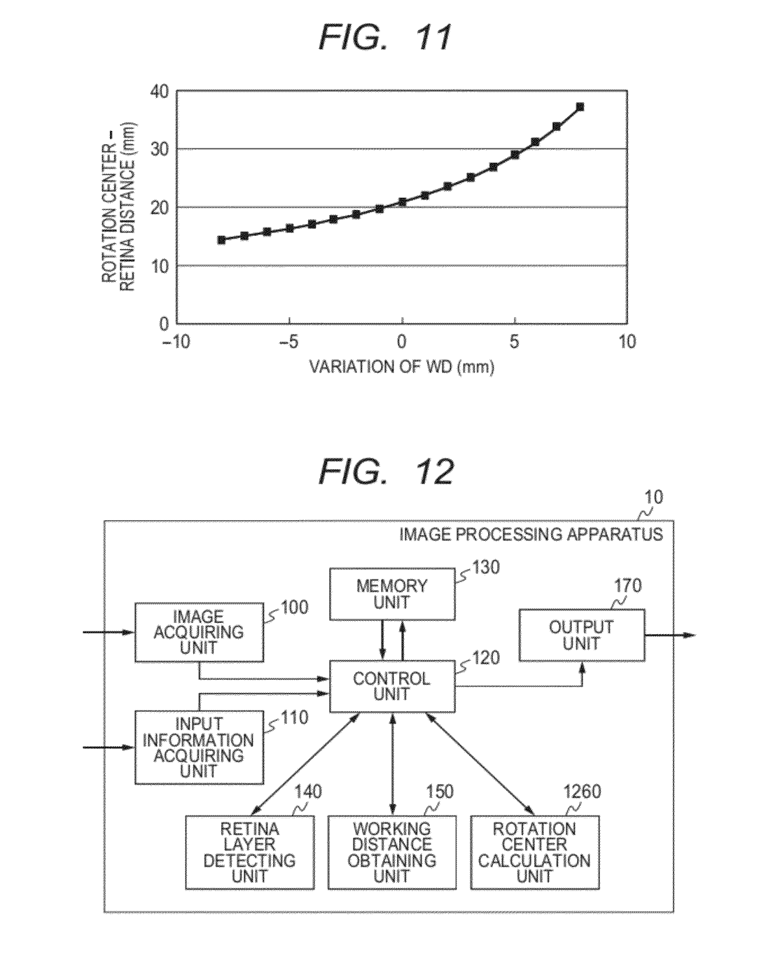

Next, a positional relationship between a variation of the working distance and the effective rotation center 202 is described. As shown in a simulation result of FIG. 11, a distance from the rotation center 202 to the retina 125 is not proportional to the variation of the working distance. This is because the light is refracted at the cornea and at the crystalline lens. In FIG. 11, a horizontal axis indicates the variation of the working distance from the pupil, and a vertical axis indicates the distance from the rotation center 202 to the retina 125. If the working distance is smaller than the design value, it is understood that a movement of the rotation center is smaller than the variation of the working distance. On the other hand, if the working distance is larger than the design value, it is understood that the movement of the rotation center is larger than the variation of the working distance.

(Step S262)

In Step S262, the image correcting unit 160 performs conversion of the acquired image into a spatial image based on the rotation center calculated in Step S261. FIG. 5C is a schematic diagram illustrating the coordinate conversion.

The tomographic image is acquired by the OCT apparatus by generating a rectangular image from a signal acquired in a sector region from the coherence gate position 126 like concentric circles, with the effective pivot position 202 as the center (FIG. 5C). In FIG. 5C, unlike FIG. 5B, the effective pivot position 202 is regarded as the origin of coordinates, a retina direction is regarded as a z axis, and a direction corresponding to a scan angle is regarded as an x axis.

In the tomographic image, the upper left corner is regarded as the origin, the number of pixels from the origin in a depth direction of the retina is denoted by j, and the number of pixels from the origin in a direction parallel to the retina is denoted by i. A size of the image is Nₕ in the depth direction and N_{w} in the width direction. In addition, a scan angle viewed from the retina is denoted by Θ.

Positions on the j-th row in the image are positions having the same distance from the coherence gate, and hence the following relationships are satisfied.

$$\begin{matrix}{x}^{2} + {z}^{2} = ( L + \frac{jh}{n} ) & Equation 11 \\ x = - z tan ( Θ \frac{{N}_{w} - 2 i}{2 {N}_{w}} ) & Equation 12\end{matrix}$$

In Equation 11, L denotes an actual distance from the effective pivot position to the coherence gate position, h denotes a pixel resolution in the retina depth direction, and n denotes a refractive index. In Equation 11, the upper end of the image is the coherence gate position.

When relationships of Equations 11 and 12 are used, the position on the space coordinates of the pixel (i, j) on the image is expressed as follows.

$$\begin{matrix}z = ( L + \frac{jh}{n} ) cos ( Θ \frac{{N}_{w} - 2 i}{2 {N}_{w}} ) & Equation 13 \\ x = - ( L + \frac{jh}{n} ) sin ( Θ \frac{{N}_{w} - 2 i}{2 {N}_{w}} ) & Equation 14\end{matrix}$$

(Step S263)

In Step S263, the image correcting unit 160 forms the corrected image based on the conversion into the spatial image in Step S262. Then, the formed corrected image is stored in the memory unit 130 via the control unit 120.

The corrected image is formed by reflecting the actual position of the region in which the tomographic image is acquired. However, as illustrated in FIG. 5C, the region becomes a sector. Therefore, the region is expanded so that the region in which the tomographic image is acquired is substantially included, and the image is formed. In this case, it should be noted that the resolution of the OCT tomographic image is different between the direction parallel to the retina (horizontal direction in the image) and the depth direction of the retina (vertical direction in the image). This difference is provided for the purpose of acquiring more information in the depth direction of the retina, and therefore the image is enlarged for display in the vertical direction. When the corrected image is formed, this aspect ratio is maintained and saved. This is because the detailed information in the depth direction of the retina is important for medical doctors.

An example of a method of forming the corrected image is described below. A size of the tomographic image to be an input image is Nₕ in the vertical direction and N_{w} in the horizontal direction, and a field angle for acquiring an image is 9 mm. A pixel on the tomographic image is expressed by (i, j). In contrast, a pixel on the corrected image is expressed by (m, l), and a size thereof is the same as the tomographic image, namely, Nₕ in the vertical direction and N_{w} in the horizontal direction. However, a region included in the corrected image is larger than the region of the tomographic image, which has a width W of approximately 10 mm and the height H is set so as to maintain the aspect ratio when the image is acquired. In addition, conversion is performed so that the center of the acquired image (N_{w}/2, Nₕ/2) becomes the center of the corrected image.

First, the space coordinates of the center of the tomographic image are set to (0, z_{center}).

$${z}_{center} = L + \frac{M}{2} \frac{h}{n} \qquad Equation 15$$

The space coordinates (x, z) corresponding to the pixel (m, l) on the corrected image are described as follows.

$$\begin{matrix}x = W ( \frac{m}{{N}_{w}} - \frac{1}{2} ) & Equation 16 \\ z = {z}_{center} + H ( \frac{l}{{N}_{w}} - \frac{1}{2} ) & Equation 17\end{matrix}$$

The pixel on the tomographic image corresponding to the space coordinates (x, z) is expressed as follows as reverse conversions of Equations 13 and 14.

$$\begin{matrix}i = \frac{{N}_{w}}{2} + \frac{{N}_{w}}{Θ} arc tan ( \frac{x}{z} ) & Equation 18 \\ j = L \frac{n}{h} - \sqrt{{( \frac{n}{h} x )}^{2} + {( \frac{n}{h} z )}^{2}} & Equation 19\end{matrix}$$

Values of (i, j) determined by Equations 18 and 19 are real values. The real values are rounded down or up to be integers, and linear interpolation of four pixels on the tomographic image is performed to calculate pixel values of the pixel (m, l) on the corrected image.

(Step S270)

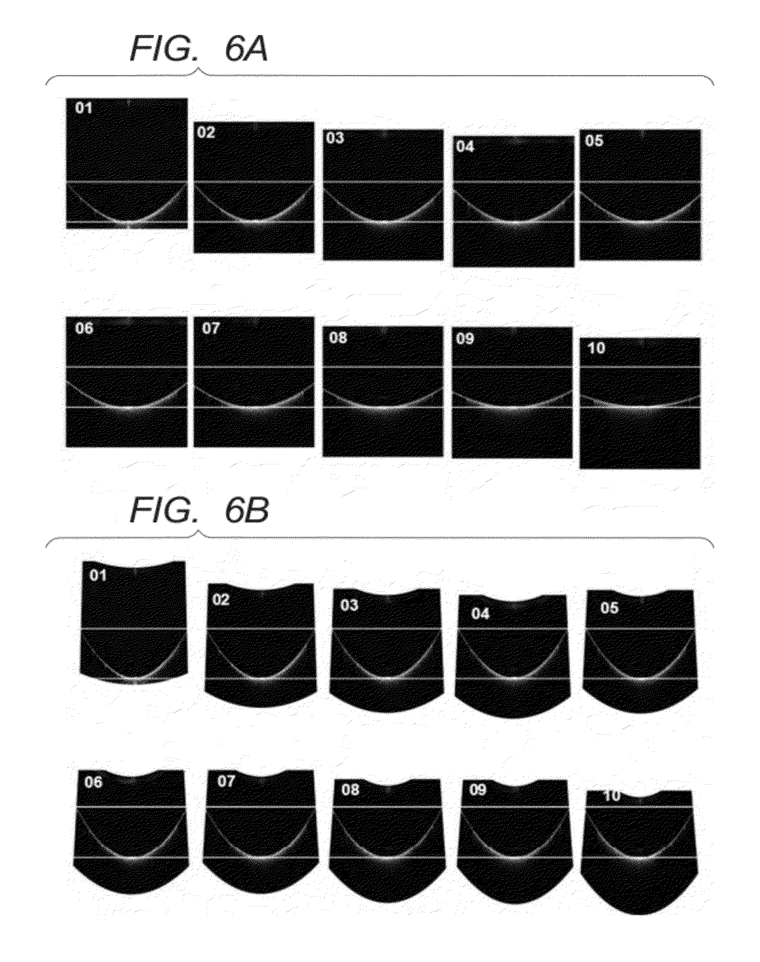

In Step S270, the output unit 170 displays the corrected image generated in Step S260 on the monitor via the output unit 170. FIGS. 6A and 6B show an example of the acquired tomographic image and the corrected image thereof. FIG. 6A shows tomographic images of the same model eye acquired while changing the working distance. The working distance is largest in the image having the number 01, and the working distance becomes smaller (push amount becomes larger) as the number increases. As shown in FIG. 6A, it is understood that the curvature on the image varies as the working distance varies.

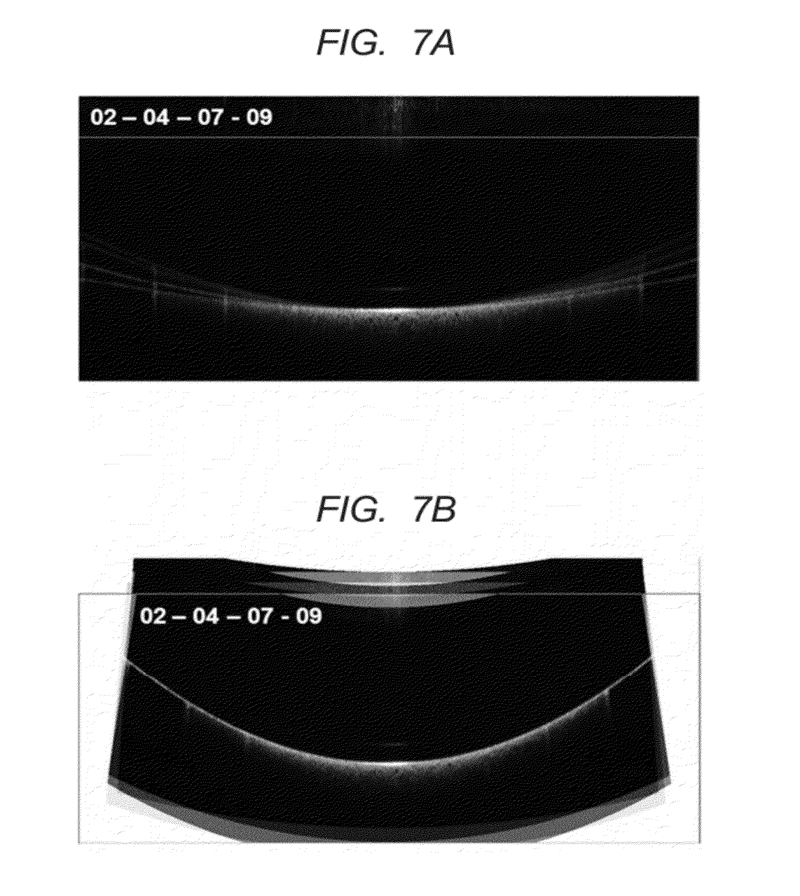

FIG. 6B shows corrected images of the images of FIG. 6A according to this embodiment. In addition, FIG. 7A shows an image obtained by superimposing the images shown in FIG. 6A, and FIG. 7B shows an image obtained by superimposing the corrected images. In this embodiment, the corrected image is formed by increasing the field angle so that a change of the shape can be easily viewed. As shown in FIG. 6B, the curvature is substantially constant in the corrected image.

(Step S280)

In Step S280, the output unit 170 stores the information of the eye to be inspected and the working distance calculated based on the information, which are acquired in Steps S210 to Step S250, in the database.

With the configuration described above, when the image of the retina is acquired by the OCT apparatus, it is possible to display the image after correcting the difference of curvature due to the difference of working distance when the image is acquired, and thus there is an effect that the difference of curvature among different eyes to be inspected and a variation of the curvature in the same eye to be inspected can be compared in a quantitative manner.

### Second Embodiment

In a second embodiment of the present invention, when acquiring the working distance when the image is acquired by the method described above in the first embodiment, the acquired working distance is compared with the working distance of the image that has been acquired before for the same eye to be inspected, and a difference between the working distances is displayed so as to assist the operator to acquire an image at the same working distance. The first embodiment describes that it is possible to correct the image to have the same curvature even if the images are acquired at different working distances. However, in general, image quality of the corrected image is deteriorated from that of the original tomographic image. Therefore, if the image is acquired at a working distance as close as possible to the working distance when the image was acquired last time, it is possible to compare the retina state between images without correction.

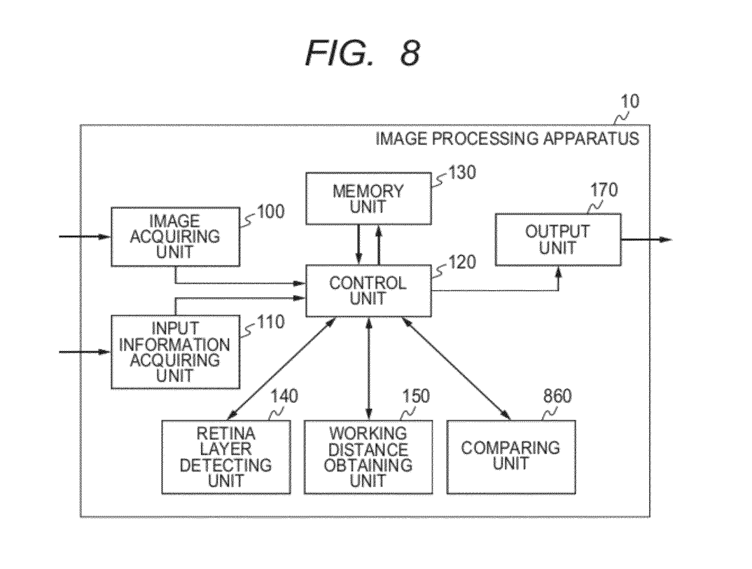

A functional configuration of the image processing apparatus 10 according to this embodiment is illustrated in FIG. 8. However, portions other than a comparing unit 860 of FIG. 8 are the same as those illustrated in FIG. 1, and hence descriptions thereof are omitted. The comparing unit 860 compares the working distance calculated from the acquired tomographic image with the working distance when the image was acquired last time. In this case, the working distance when the image was acquired last time is regarded as a reference working distance to be a reference for the working distance of acquiring the image this time. Note that, the reference working distance may remain the same as the working distance when the image was acquired last time or may be set based on the working distance of the last time, for example, by multiplying the working distance of the last time by a predetermined coefficient. Further, as described above, the working distance in which the distance from the rotation center to the retina remains the same as that of the last time the image is acquired may be regarded as the reference working distance.

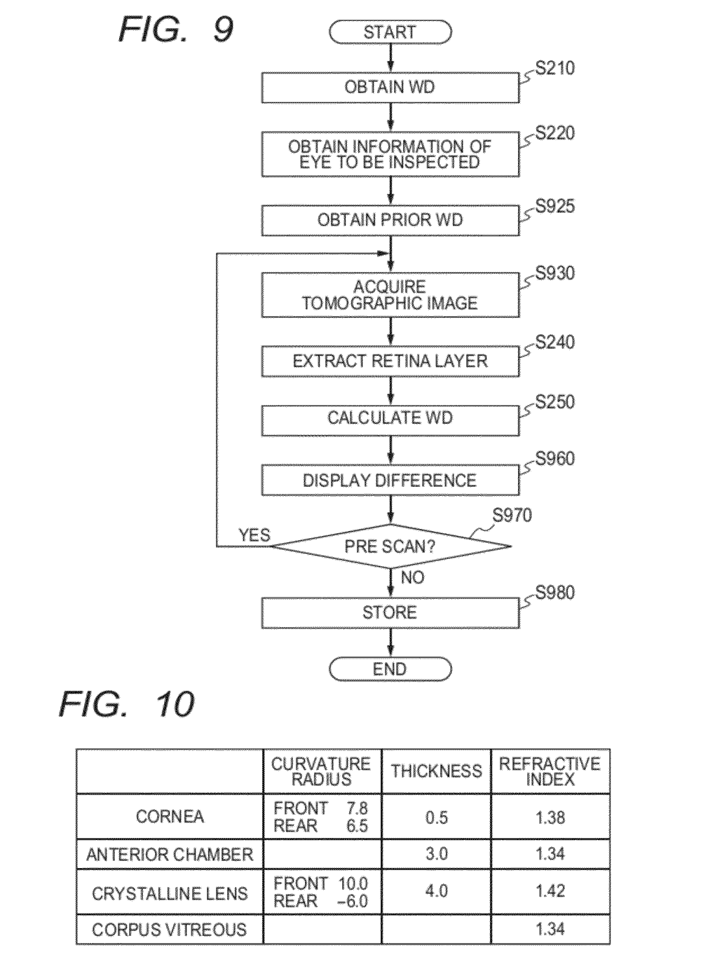

Next, referring to a flowchart of FIG. 9, a process procedure of the image processing apparatus 10 of this embodiment is described. In this embodiment, the process procedure of Steps S210, S220, S240, and S250 is the same as that described in the first embodiment, and hence description thereof is omitted. However, in contrast to the first embodiment in which the curvature of the acquired tomographic image is corrected after acquiring the image, it is necessary in this embodiment to inform the operator so that the working distance when acquiring the image becomes the same as that when the image was acquired last time. Therefore, the process procedure of Steps S210 to S250 is the same as that described in the first embodiment, but the tomographic image acquired in Step S930 in this embodiment is different from that in the first embodiment. Specifically, the tomographic image acquired in Step S930 of this embodiment is a prescan image before acquiring the image while the tomographic image acquired in Step S930 of the first embodiment is the acquired tomographic image. Herein, the prescan image refers to an image acquired at high speed by rough sampling, which is displayed to the operator for performing adjustment of the coherence gate position or the focus. In order to distinguish this prescan image, the tomographic image acquired regularly is referred to as a regular scan image.

In this embodiment, when the coherence gate adjustment or the like is performed in the prescan, the working distance of the acquired prescan image is calculated and displayed in real time.

(Step S925)

In Step S925, the input information acquiring unit 110 acquires the working distance when the image was acquired last time for the same eye to be inspected from the database based on the information of the eye to be inspected stored in the memory unit 130. Then, the acquired working distance is stored in the memory unit 130 via the control unit 120.

(Step S930)

In Step S930, the image acquiring unit 100 acquires the tomographic image from the OCT apparatus connected to the image processing apparatus 10. Then, the acquired tomographic image is stored in the memory unit 130 via the control unit 120.

In addition, in this step, the coherence gate position 126 when the acquired image is acquired, and information as to whether the acquired tomographic image is acquired as the prescan image or as the regular scan image is acquired and stored in the memory unit 130 via the control unit 120.

(Step S960)

In Step S960, the comparing unit 860 compares the working distance when the image was acquired last time, which is acquired in Step S925, with the working distance when the prescan image is acquired, which is acquired in Step S250, and a difference between the working distances is calculated. Then, the calculated difference is displayed on the monitor (not shown) via the output unit 170.

There are considered various methods of displaying a difference between the working distance when the prescan image is acquired and that when the image was acquired last time. For instance, there is considered a method of providing a difference value on the display screen of the prescan image as a positive value if the working distance is larger than that when the image was acquired last time, or as a negative value if the working distance is smaller than that when the image was acquired last time. In this case, if the difference value is within a range of plus/minus 1 mm, the difference value may be displayed in blue, and if the difference value is out of the range, the difference value may be displayed in red or in a larger size, so as to warn the operator effectively.

(Step S970)

In Step S970, the control unit 120 branches the process based on information whether or not the acquired image acquired in Step S930 is the prescan image. When the acquired image is the prescan image, the process returns back to Step S930 in which a next image is acquired and the same process is repeated. When the acquired image is the regular scan image, the process proceeds to Step S980.

(Step S980)

In Step S980, the output unit 170 stores the information of the eye to be inspected and the working distance calculated based on the information, which are acquired in Steps S210 to S960, in the database.

In other words, in the embodiment described above, a result of acquiring the image of the eye to be inspected performed by using the reference working distance is displayed on a display unit, and further a display form indicating that the assistance to acquire the image has been performed using the reference working distance is displayed together. With the configuration described above, when the coherence gate and the focus are adjusted before acquiring the image of the retina by the OCT apparatus, adjustment of the working distance can be performed simultaneously. The image is acquired at the working distance that is close to that when the image was acquired last time, and hence it is possible to obtain an effect that the retina state can be compared not between the corrected images in which image quality is generally deteriorated but between the images without correction. Note that, the operation of causing the display unit to display a display form in which the assistance to acquire the image of the eye to be inspected is being performed at the reference working distance which is set based on the working distance is performed by a unit functioning as a display control unit in the control unit 120 in the present invention.

### Third Embodiment

In the second embodiment, description is given of a method in which the image is acquired at the same working distance as that when the image was acquired last time, so as to enable comparison of the retina including the curvature between the images without the correction, too. However, in a myopic eye, when inspecting an eye in which progress of posterior staphyloma or the like is observed, there may be a case where elongation of the axial length is observed in a progress observation. In this case, if the working distance is equalized (supposing that there is no change with time in the anterior eye part), the rotation center for scanning of the measuring light can be positioned at a position of the same positional relationship from the anterior eye part. However, the distance from the rotation center to the retina is changed when the axial length is elongated, and hence it is difficult to compare between the obtained images even if the coherence gate position is adjusted.

A third embodiment of the present invention has a feature that in order to note a structure of the retina in the macula lutea and its vicinity, the rotation center is adjusted so that the distance from the macula lutea is equalized in the acquired image having the macula lutea as its center. The information of how much the working distance of the current prescan image is shifted from the reference of the working distance in which the distance from the macula lutea is equal to that when the image was acquired last time is provided to the operator, and hence the operator is assisted in adjusting the working distance.

A functional configuration of the image processing apparatus 10 according to this embodiment is illustrated in FIG. 12. However, portions other than a rotation center calculating portion 1260 of FIG. 12 are the same as those illustrated in FIG. 1, and hence descriptions thereof are omitted. The rotation center calculating portion 1260 calculates a distance between the rotation center and the retina based on the working distance calculated from the acquired tomographic image. This corresponds to an operation of Step S261 in the first embodiment. Then, a difference between the working distance in a case where the distance from the rotation center to the retina is the same as that when the image was acquired last time and the working distance in a target image are determined.

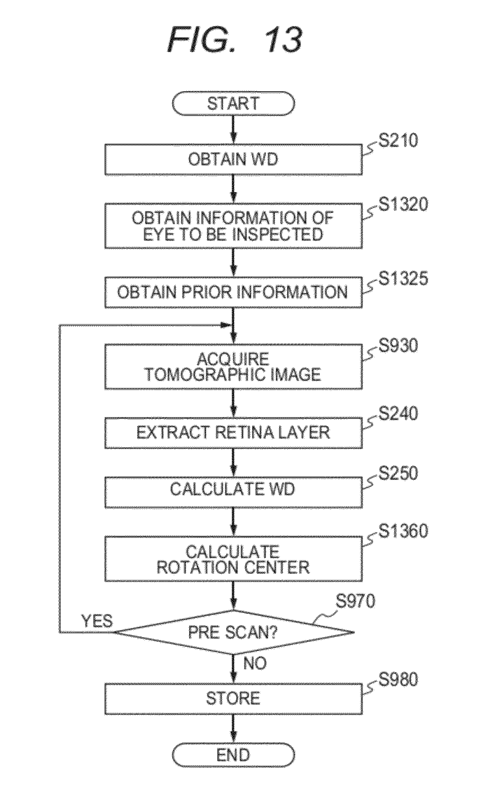

Further, referring to a flowchart of FIG. 13, a process procedure of the image processing apparatus 10 in this embodiment is described. In the following, descriptions of the same steps as in the first and second embodiments are omitted, and only different Steps S1320, S1325, and S1360 are described in detail.

In addition, in this embodiment, similarly to the second embodiment, when the adjustment or the like of the coherence gate position is performed in the prescan, the rotation center is calculated in real time from the acquired prescan image, and the display based on the value is performed. A distance between the rotation center and the retina is calculated from the calculated value of the rotation center, and a display is performed so as to prompt the operator to adjust the working distance so that the distance between the rotation center and the retina becomes the same as that when the image was acquired last time.

(Step S1320)

In Step S1320, the input information acquiring unit 110 acquires information of the eye to be inspected based on the database or an input from the input portion (not shown) by the operator. Herein, the information of the eye to be inspected refers to eye parameters of the eye to be inspected, which are typified by the axial length and a curvature of the cornea, and the acquired information is stored in the memory unit 130 via the control unit 120.

Next, the rotation center calculating portion 1260 determines a relationship between the working distance and the rotation center based on the acquired eye parameters of the eye to be inspected by using the same method as in Step S261. An example of the obtained result is shown in FIG. 11, and this result is stored in the memory unit 130.

In this step, if the image is acquired with the macula lutea as the center, a distance from the retina to the rotation center can be regarded as a distance from the macula lutea to the rotation center.

(Step S1325)

In Step S1325, the input information acquiring unit 110 acquires the information when the image was acquired last time for the same eye to be inspected from the database based on the information of the eye to be inspected stored in the memory unit 130. Herein, the information when the image was acquired last time refers to the axial length, the working distance, the rotation center, the distance from the retina to the rotation center, and the like, which are obtained when the image was acquired last time. Then, the acquired information is stored in the memory unit 130 via the control unit 120.

(Step S1360)

In Step S1360, the rotation center calculating portion 1260 acquires the distance from the retina to the rotation center when the image was acquired last time, which is acquired in Step S1325. Then, through use of the relationship between the working distance and the distance from the rotation center to the retina, which is obtained in Step S1320, the working distance is calculated, in which the rotation center is at the same distance from the retina as that when the image was acquired last time.

The calculated working distance is compared with the working distance of the current acquired image calculated in Step S250, and the difference between the working distances is displayed in the monitor (not shown) via the display portion 170.

With the configuration described above, in the case where the axial length is elongated in the progress observation, it is possible to acquire the image by adjusting the working distance so that the distance from the retina, particularly from the macula lutea center to the rotation center becomes the same as that when the image was acquired last time. This configuration provides an effect that, even if the eye to be inspected is not the same, the retina state in a vicinity of the macula lutea can be observed in the same condition by acquiring the image with the rotation center positioned at always the same distance from the macula lutea.

### Another Embodiment

It is to be understood that an object of the present invention can be achieved by a configuration in which a storage medium storing program codes of software for realizing the functions of the above-mentioned embodiments is supplied to a system or an apparatus, and a computer (or a CPU or an MPU) of the system or the apparatus reads out the program codes stored in the storage medium and executes the program codes.

### Still Another Embodiment

In addition, the present invention can also be realized by performing the following process. Specifically, in the process, the software (program) for realizing the functions of the above-mentioned embodiments is supplied to the system or the apparatus via a network or various storage media, and a computer (or a CPU or an MPU) of the system or the apparatus reads out the program and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-132328, filed Jun. 14, 2011, which is hereby incorporated by reference herein in its entirety.

What is claimed is:

1. An ophthalmologic apparatus, comprising:
an image acquiring unit configured to acquire a tomographic image of a fundus of an eye to be inspected;
a calculating unit configured to calculate a working distance, based on a predetermined layer of the tomographic image, a coherence gate position, and an axial length of the eye to be inspected, at a time when the tomographic image is acquired; and
a correcting unit configured to correct the tomographic image based on the working distance.

2. An ophthalmologic apparatus according to claim 1, further comprising:
a layer extracting unit configured to extract the predetermined layer from the tomographic image; and
an obtaining unit configured to obtain a retina distance as a distance from the coherence gate position to the predetermined layer when the tomographic image is acquired,
wherein the calculating unit calculates the working distance based on the retina distance, the coherence gate position, and the axial length of the eye to be inspected.

3. An ophthalmologic apparatus according to claim 2, further comprising an eye information acquiring unit configured to acquire a distance from a reference position to the coherence gate position, and the axial length of the eye to be inspected,
wherein the calculating unit calculates the working distance based on the retina distance, the distance from the reference position to the coherence gate position, and the axial length of the eye to be inspected.

4. An ophthalmologic apparatus according to claim 1, further comprising a display control unit configured to control a display unit to display a display form indicating execution of assistance to acquire an image of the eye to be inspected at a reference working distance set based on the working distance.

5. An ophthalmologic apparatus according to claim 4, wherein the reference working distance is one of the same working distance as that when the image was acquired last time and a working distance in which a distance from a rotation center of measuring light scanned by a scanning unit to a retina is the same as that when the image was acquired last time.

6. An ophthalmologic apparatus according to claim 1, further comprising an objective lens,
wherein the calculating unit calculates a distance from the objective lens to the eye to be inspected as the working distance.

7. An ophthalmologic system, comprising:
an image acquiring unit configured to acquire a tomographic image of a fundus of an eye to be inspected;
a calculating unit configured to calculate a working distance, based on a predetermined layer of the tomographic image, a coherence gate position, and an axial length of the eye to be inspected, at a time when the tomographic image is acquired; and
a correcting unit configured to correct the tomographic image based on the working distance.

8. An image processing apparatus, comprising:
an image acquiring unit configured to acquire a tomographic image of a fundus of an eye to be inspected;
a calculating unit configured to calculate a working distance, based on a predetermined layer of the tomographic image, a coherence gate position, and an axial length of the eye to be inspected, at a time when the tomographic image is acquired; and
a correcting unit configured to correct the tomographic image based on the working distance.

9. An image processing method, comprising:
acquiring a tomographic image of a fundus of an eye to be inspected;
calculating a working distance, based on a predetermined layer of the tomographic image, a coherence gate position, and an axial length of the eye to be inspected, at a time when the tomographic image is acquired; and
correcting the tomographic image based on the working distance.

10. A program for causing a computer to execute the image processing method according to claim 9.
